(19) United States
(12) Patent Application Publication
(10) Pub. No.: US 2021/0401727 A1
(43) Pub. Date: Dec. 30, 2021

(54) AEROSOLIZED COMPOSITIONS, SYSTEMS, KITS, AND METHODS FOR KERATINOUS SUBSTRATES

(71) Applicant: L'Oreal, Paris (FR)

(72) Inventors: Anna BOTTO, Cranford, NJ (US); Emma NAIBERK, Maplewood, NJ (US); Pedro APRIGLIANO FERNANDES, Rio de Janeiro (BR)

(21) Appl. No.: 16/915,929

(22) Filed: Jun. 29, 2020

(51) Int. Cl. A61K 8/92; A61K 8/34; A61K 8/42; A61Q 5/12; A61K 8/04

(52) U.S. Cl. CPC A61K 8/922; A61K 8/342; A61K 8/42; A61K 2800/5426; A61K 8/046; A61K 2800/87; A61K 2800/10; A61Q 5/12

(57) ABSTRACT

The disclosure relates to compositions for treating, caring for, and/or conditioning keratinous substrates, the compositions comprising (a) at least one vegetable oil; (b) at least one fatty alcohol; (c) at least one cationic conditioning agent; and (d) at least one propellant, as well as systems and kits comprising the compositions, and methods of using the compositions.

### TECHNICAL FIELD

The present disclosure relates to compositions for treating, caring for, and/or conditioning keratinous substrates, as well as systems and kits comprising the compositions, and methods of using the compositions.

### BACKGROUND

Consumers desire new and improved compositions for treating, caring for, and/or conditioning keratin substances, such as skin or hair. It is understood that different forms of compositions can provide different benefits, yet can also present different drawbacks and/or challenges, such as, for example, formulation challenges.

For example, hair care compositions, such as hair conditioner and/or treatment compositions, may be used before or after the hair has been washed with shampoo and/or subjected to a chemical treatment to improve or return to the hair its natural luster, shine, and softness, or to improve the feel, appearance, and manageability of hairs. Traditional hair conditioning or treatment compositions may be rinse-off or leave-on cream-like emulsions or lotions. However, while generally allowing for ease of formulation, certain components in such traditional hair treatment and/or conditioner compositions (e.g. silicones and/or ethanol) may cause build-up on the hair and/or may cause the hair to feel heavy or weighed-down, and/or may not provide sufficient conditioning benefits such as smoothness, shaping, and/or moisture effect. Additionally, the presence of silicones in a composition may present difficulties in preparing the compositions in various formulations, e.g. as an aerosolized foam.

It is therefore desired to provide new and improved compositions for treating, caring for, and/or conditioning keratin substances, such as skin or hair, which may avoid or minimize formulation challenges yet provide benefits to the keratin substrates to which the compositions are applied.

It has now surprisingly been found that aerosolized foam compositions described herein provide a treatment, care, and/or conditioning composition that is in the form of a fluid emulsion with low viscosity prior to being aerosolized, which, when aerosolized, forms a compact, dense, and supple foam. Additionally, it has been found that aerosolized foam compositions according to the disclosure are surprisingly and unexpectedly more stable than other hair or skin care aerosol foams, and do not collapse. In addition, the compositions provide improved properties to the keratin substrates relative to traditional care, treatment, and/or conditioning compositions. Optionally, the compositions may be free of ethanol and silicones. The ingredients in the compositions may optionally be sustainable, i.e. of natural and/or organic sourcing.

### SUMMARY

The disclosure relates to aerosolized foam compositions useful for treating, caring for, and/or conditioning the skin and/or hair, as well as systems and kits comprising the compositions, and methods of using the compositions. In various exemplary embodiments, the aerosolized foam compositions may be hair treatment, care, and/or conditioner compositions, for example rinse-off or leave-on skin or hair compositions that may be used either before or after other skin or hair treatment or care compositions or processes. The aerosolized foam compositions may, in further embodiments, be hair-color-toning compositions, or compositions for depositing color onto hair. In various embodiments, the aerosolized foam compositions may deliver one or more property such as smoothness, conditioning, ease of shaping, and/or moisturized look and feel to the hair. In various embodiments, the aerosolized foam compositions may allow for ease of application and/or distribution of the composition to the hair.

In one exemplary and non-limiting embodiment, the disclosure relates to compositions for treating keratinous substrates, the compositions comprising (a) at least one vegetable oil, (b) at least one liquid fatty alcohol, (c) at least one cationic conditioning agent, and (d) at least one propellant, wherein the composition is in the form of a fluid emulsion. For example, the compositions may comprise (a) at least one vegetable oil, (b) at least one liquid fatty alcohol, (c) at least one cationic conditioning agent, (d) at least one propellant, (e) at least one solid fatty alcohol, and (f) at least one component chosen from direct dyes, pigments, or combinations thereof.

In a further exemplary and non-limiting embodiment, the disclosure relates to compositions for treating keratinous substrates, the compositions comprising (a) coconut oil in an amount ranging from about 0.5% to about 5%, (b) octyldodecanol, (c) at least one amidoamine, (d) at least one propellant, (e) at least one solid fatty alcohol, and (f) optionally at least one additional component chosen from active agents, direct dyes, pigments, surfactants, or mixtures thereof, wherein the composition is in the form of a fluid emulsion.

In a further exemplary and non-limiting embodiment, the disclosure relates to systems comprising an aerosol device, the aerosol device containing a composition comprising (a) at least one vegetable oil, (b) at least one liquid fatty alcohol, (c) at least one cationic conditioning agent, (d) at least one propellant, (e) optionally at least one solid fatty alcohol, and (f) optionally at least one additional component chosen from active agents, direct dyes, pigments, surfactants, or mixtures thereof, wherein the composition is in the form of a fluid emulsion, and wherein the aerosol device comprises a means for dispensing the content of the container.

In yet a further exemplary and non-limiting embodiment, the disclosure relates to methods of treating, caring for, or conditioning a keratin substrate, the methods comprising applying to the keratin substrate an aerosolized foam composition, wherein the aerosolized foam composition comprises (a) at least one vegetable oil, (b) at least one liquid fatty alcohol, (c) at least one cationic conditioning agent, (d) at least one propellant, (e) optionally at least one solid fatty alcohol, and (f) optionally at least one additional component chosen from active agents, direct dyes, pigments, surfactants, or mixtures thereof, and optionally rinsing the composition from the keratin substrate.

In a still further exemplary and non-limiting embodiment, the disclosure relates to kits comprising an aerosol container containing a first composition, the first composition comprising (a) at least one vegetable oil, (b) at least one liquid fatty alcohol, (c) at least one cationic conditioning agent, (d) at least one propellant, (e) optionally at least one solid fatty alcohol, and (f) optionally at least one additional component chosen from active agents, direct dyes, pigments, surfactants, or mixtures thereof, wherein the composition is in the form of a fluid emulsion, and a second container comprising a second composition for treating, caring for, or conditioning keratin substrates.

### BRIEF DESCRIPTION OF THE DRAWINGS

The accompanying drawings, which are incorporated herein and constitute part of this specification, illustrate exemplary embodiments of the invention, and, together with the general description given above and the description provided herein, serve to explain features of the invention.

FIG. 1 is a photographic image depicting an exemplary foam hair-color-toning composition according to one embodiment of the disclosure.

FIG. 2 is a photographic image depicting an exemplary foam conditioner according to one embodiment of the disclosure in comparison with a conventional non-aerosolized cream conditioner.

FIG. 3 is a photographic image depicting two compositions that differ from each other by the inclusion or exclusion of coconut oil, in a form prior to being aerosolized.

FIGS. 4A-4E are photographic images comparing the stability of a foam hair conditioner according to the disclosure with a commercial foam hair conditioner over time.

It is to be understood that the foregoing and following descriptions are exemplary and explanatory only, and are not intended to be restrictive of any subject matter claimed.

### DESCRIPTION

The present disclosure relates to compositions comprising at least one vegetable oil, at least one fatty alcohol, at least one cationic conditioning agent, and at least one propellant. The compositions may be in the form of a low-viscosity fluid emulsion prior to being aerosolized, or may be in the form of aerosol foam compositions. The present disclosure further relates to system and kits comprising the compositions, and methods of using the aerosolized foam compositions.

### I. Compositions

The compositions according to the disclosure comprise at least one vegetable oil, at least one fatty alcohol, at least one cationic conditioning agent, and at least one propellant. In certain embodiments, the compositions may be free or substantially free of ethanol and/or silicones.

In various exemplary and non-limiting embodiments, the compositions may be in the form of a fluid emulsion with low viscosity prior to being aerosolized and when aerosolized, form compact, dense, and/or stable foams that do not collapse and do not look “airy” or “whipped.” The aerosolized foam compositions may, in at least some embodiments, impart one or more properties such as ease of distribution, ease of rinsing, suppleness, softness, shine, moisturizing effect, smoothness, conditioning, detangling, shaping, frizz control, discipline, overall feel of the hair, body, volume, fullness, movement, and/or bounce to the hair. The aerosolized foam compositions may, in further embodiments, be useful as hair-color-toning compositions, or as compositions for depositing color onto hair.

### Vegetable Oil

Compositions according to the disclosure comprise at least one vegetable oil. Vegetable oils are oils derived from a plant, for example, oils from seeds or fruits. A high level of vegetable oil, such as an amount of about 1% or higher, is not typically included in traditional emulsion cosmetic compositions because oils at this level may negatively impact the stability of the emulsion cosmetic composition. However, it has been found that including vegetable oil in the compositions according to the disclosure provides increased beneficial conditioning properties to keratinous substrates, and, when combined with other components in the compositions, even at a relatively high concentration, surprisingly and permits aerosolization of the emulsion to provide a stable aerosolized foam composition.

Vegetable oils that may be used according to the disclosure include, but are not limited to, coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and mixtures thereof. In various embodiments, compositions according to the disclosure comprise coconut oil. In further embodiments, compositions according to the disclosure are substantially free of vegetable oils other than coconut oil. Thus, in various embodiments, the at least one vegetable oil may comprise, consist essentially of, or consist of coconut oil.

The total amount of vegetable oil that may be included in various embodiments may range from about 0.01% to about 15%, such as from about 0.01% to about 10%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1.5%, about 0.01% to about 1%, about 0.1% to about 15%, about 0.1% to about 10%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1.5%, about 0.1% to about 1%, about 0.5% to about 15%, about 0.5% to about 10%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1.5%, or about 0.5% to about 1%, based on the total weight of the composition. In certain embodiments, vegetable oil is present in an amount of at least about 0.1% by weight, such as at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, or at least about 2% by weight, such as about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% by weight, relative to the total weight of the composition.

### Fatty Alcohol

Compositions according to the disclosure comprise at least one fatty alcohol. In certain embodiments, the compositions comprise at least two fatty alcohols. The at least one fatty alcohol may, in some embodiments, be chosen from liquid fatty alcohols. In further embodiments, the at least one fatty alcohol may be chosen from combinations of at least one liquid fatty alcohol and at least one solid fatty alcohol.

As used herein, “fatty alcohol” refers to any alcohol with a carbon chain of C5 or greater, such as, for example, C8 or greater, C10 or greater, and C12 or greater. The fatty alcohols useful according to the disclosure include, but are not limited to, alkoxylated or non-alkoxylated, saturated or unsaturated, linear or branched, fatty alcohols, for example with from 6 to 30 carbon atoms, such as from 8 to 30 carbon atoms.

As used herein, “alkoxylated fatty alcohol” refers to any fatty alcohol with a carbon chain of C5 or greater, as defined above, further comprising at least one alkoxy group. For example, the at least one alkoxylated fatty alcohol may have a carbon chain of C8 or greater, C10 or greater, and C12 or greater. Further, for example, the at least one alkoxylated fatty alcohol may be chosen from alkoxylated polymers (including co-, ter- and homo-polymers) derived from alcohols such as glycerol (e.g. polyglyceryl derived from four glycerol molecules). The at least one alkoxy group of the at least one alkoxylated fatty alcohol may, for example, be derived from an alkoxylation reaction carried out with alkylene oxide. Non-limiting examples of at least one alkoxylated fatty alcohol include any fatty alcohol comprising at least one polyethylene glycol ether and any fatty alcohol comprising at least one polypropylene glycol ether.

According to various embodiments, the at least one fatty alcohol is chosen from liquid fatty alcohols, in particular those containing C10-C34, may have branched carbon chains and/or have branched and/or unsaturated (C═C double bond), and contain from 12 to 40 carbon atoms. The liquid fatty alcohols may be represented by the formula R—OH, wherein R denotes a C12-C24 branched alkyl group or an alkenyl group (comprising at least one C12-C24 double bond C═C), R being optionally substituted by one or more hydroxy groups. In some embodiments, the liquid fatty alcohol is a branched saturated alcohol. In at least certain embodiments, R does not contain a hydroxyl group. The liquid fatty alcohol may be chosen from oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

According to further embodiments, the at least one fatty alcohol is chosen from solid fatty alcohols. Solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm. The solid fatty alcohols may be soluble, under the same temperature and pressure conditions, in at least one organic solvent (for example ethanol, chloroform, benzene or liquid petroleum jelly) to at least 1% by weight.

The solid fatty alcohols may be represented by the formula R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

For example, the at least one solid fatty alcohol may be chosen from lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol); or mixtures thereof. In some embodiments, the compositions disclosed herein comprise at least one solid fatty alcohol chosen from cetyl alcohol, stearyl alcohol, behenyl alcohol and mixtures thereof such as cetylstearyl or cetearyl alcohol.

Thus, without intending to be limiting, the at least one fatty alcohol may be chosen from C9-C11 alcohols, C12-C13 alcohols, C12-C15 alcohols, C12-C16 alcohols, C14-C15 alcohols, C12-C22 alcohols, arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, 2-octyldodecanol, isostearyl alcohol, 2-hexyldecanol, 2-heptyldecanol, 2-octyldecanol, caproic alcohol (1-hexanol), enanthic alcohol (1-heptanol), caprylic alcohol (1-octanol), pelargonic alcohol (1-nonanol), capric alcohol (1-decanol), lauryl alcohol (1-dodecanol), or a mixture thereof. For example, in one embodiment, the at least one fatty alcohol is octyldodecanol.

By way of example, ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, cetearel:h-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, lauretih-16, laureth-20, laureth-23, laureth-25, laureth-30, laureth-40, deceth-3, deceth-5, oleth-5, oleth-30, steareth-2, steareth-10, steareth-20, steareth-100, cetylsteareth-12, ceteareth-5, ceteareth-5, polyglyceryl 4-lauryl ether, polyglyceryl 4-oleyl ether, polyglyceryl 2-oleyl ether, polyglyceryl 2-cetyl ether, polyglyceryl 6-cetyl ether, polyglyceryl 6-oleylcetyl ether, polyglyceryl 6-octadecyl ether, C9-C11 pareth-3, C9-C11 pareth-6, C11-C15 pareth-3, C11-C15 pareth-5, C11-C15 pareth-12, C11-C15 pareth-20, C12-C15 pareth-9, C12-C15 pareth-12, C22-C24 pareth-33, or a mixture thereof may be chosen.

The total amount of fatty alcohols, or the amount of each of the individual fatty alcohols, in the composition may range up to about 15%, such from about 0.01% up to about 10% by weight, based on the total weight of the composition. By way of non-limiting example, any of the at least one fatty alcohol may be present in an amount ranging from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, or any suitable combination, sub-combination, range, or sub-range thereof by weight, based on the weight of the composition. In further embodiments, the at least one fatty alcohol may be present in an amount ranging from about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, or about 0.1% to about 1% or any suitable combination, sub-combination, range, or sub-range thereof by weight, based on the weight of the composition. Thus, any one of the at least one fatty alcohol may be present, by weight, based on the total weight of the composition, from about 0.1, 0.2, 0.3, 0.4, 0.5, 0.6, 0.7, 0.8, 0.9, 1, 2, 3, 4, 5, 6, 7, 8, 9 weight percent to about 10 weight percent, including increments and ranges therein and there between.

### Cationic Conditioning Agent

Compositions according to the disclosure comprise at least one cationic conditioning agent. In certain exemplary embodiments, the at least one cationic conditioning agent may be chosen from amidoamines.

Non-limiting exemplary useful amidoamine compounds are those corresponding to the following formula (I) and their salts:

RCONHR′N(R″)₂ (I)

wherein:

R is a linear or branched, acyclic or cyclic, saturated or unsaturated, aliphatic or aromatic, substituted or unsubstituted hydrocarbon radical containing at least 6 carbon atoms;

R′ is a linear or branched, acyclic or cyclic, saturated or unsaturated, aliphatic or aromatic, substituted or unsubstituted divalent hydrocarbon radical containing less than 6 carbon atoms, for example 2 or 3 carbon atoms; and

R″ is H or a hydrocarbon radical containing less than 6 carbon atoms.

In some embodiments, R is a linear or branched, acyclic alkyl or alkenyl group or an alkyl phenyl group. In some embodiments, R′ is a linear or branched, acyclic alkyl or alkenyl group. In some embodiments, R″ is H or a methyl group.

Examples of amidoamines that are useful in the compositions of the instant disclosure include, but are not limited to the following: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

In further embodiments, cationic conditioning agents can be chosen from monoalkyl quaternary amines, dialkyl quaternary amines, or polyquaternium compounds or salts thereof.

For example, cationic conditioning agents may be chosen from polyquaterium-(also called quaternized polyhydroxyethyl cellulose), cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, aminopropyl dimethicone, Quaterium-8, and dilinoleamidopropyl dimethylamine dimethicone PEG-7 phosphate.

In some instances, the cationic conditioning agents are cationic polymers. The term “cationic polymer” means any polymer comprising at least one cationic group and/or at least one group that may be ionized into a cationic group.

In various embodiments, cationic polymers that may be chosen include, but are not limited to: polyquaternium 4, polyquaternium 6, polyquaternium 7, polyquaternium 10, polyquaternium 11, polyquaternium 16, polyquaternium 22, polyquaternium 28, polyquaternium 32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, or guar hydroxypropyltrimonium chloride, and mixtures thereof.

However, in at least one embodiment, the aerosolized foam compositions are free of, or substantially free of, cationic conditioning agents other than amidoamines.

The total amount of the at least one cationic conditioning agent ranges from about 0.01% to about 15%, such as from about 0.01% to about 10%, about 0.01% to about 5%, about 0.01% to about 4%, about 0.01% to about 3%, about 0.01% to about 2%, about 0.01% to about 1%, about 0.1% to about 15%, about 0.1% to about 10%, about 0.1% to about 5%, about 0.1% to about 4%, about 0.1% to about 3%, about 0.1% to about 2%, about 0.1% to about 1%, about 1% to about 15%, about 1% to about 10%, about 1% to about 5%, about 1% to about 4%, about 1% to about 3%, about 2% to about 4%, or about 2% to about 3%, by weight, based on the total weight of the composition.

### Propellant

Compositions and/or devices according to the disclosure may comprise at least one propellant. In some embodiments, the propellant is in the device, but is not added to the composition, in other embodiments the propellant is added to the composition, and in yet further embodiments the propellant may be in both the composition and the device.

The at least one propellant may be chosen from any aerosol propellant, especially liquid or liquefiable propellants, suitable for being used in cosmetic or personal care compositions. For example, the at least one propellant may be chosen from any liquefiable gas or a halogenated propellant. Non-limiting examples of useful propellants include dimethyl ether and hydrocarbon propellants such as propane, n-butane, iso-butane, chlorofluorocarbons (CFCs), CFC-replacement propellants, fluorohydrocarbons, such as F 152a (1,1-difluorethane) or F 134 (tetrafluorethane), as well as propellants which are present at the pressures in question in gaseous forms such as N₂, N₂O and CO₂ as well as mixtures of the above-mentioned propellants. In some cases, particularly useful propellants include dimethyl ether, hydrofluorocarbon 152A, isobutane, propane, isopropane, butane, and a mixture thereof. For example, the propellant may be a mixture of isobutane and propane.

The total amount of the at least one propellant may vary. In some cases, the total amount of the at least one propellant may range from about 0.01% to about 10%, such as about 0.01% to about 9%, about 0.01% to about 8%, about 0.01% to about 7%, about 0.01% to about 5%, about 0.01% to about 5%, about 0.01% to about 4%, about 0.01% to about 3%, about 0.01% to about 2%, about 0.01% to about 1%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 1% to about 10%, about 1% to about 9%, about 1% to about 8%, about 1% to about 7%, about 1% to about 6%, about 1% to about 5%, about 1% to about 4%, about 1% to about 3%, or about 1% to about 2% by weight, based on the total weight of the composition. In one embodiment, the at least one propellant is present in an amount of less than or equal to about 6% by weight, relative to the total weight of the composition.

### Solvent

Compositions according to the disclosure comprise a solvent. The solvent may be chosen from water, non-aqueous solvents, or mixtures thereof.

In some embodiments, the solvent comprises, consists essentially of, or consists of water. The total amount of water in the compositions may vary depending on the type of composition and the desired consistency, viscosity, etc.

In certain embodiments, the composition comprises, consists essentially of, or consists of non-aqueous solvents, for example, glycerin, C1-4 alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some cases, the solvent may be selected from the group consisting of one or more glycols, C₁₋₄ alcohols, glycerin, and a mixture thereof. For example, the solvent may be selected from the group consisting of hexylene glycol, proplene glycol, caprylyl glycol, glycerin, isopropyl alcohol, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof.

Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The solvent may be present in the composition in an amount ranging from about 60% to about 98% by weight, relative to the total weight of the composition, including all ranges and subranges therebetween. For example, in one embodiment, the total amount of water may be about 75% to about 98%, about 75% to about 95%, about 75% to 93%, or about 75% to 90% by weight, relative to the total weight of the composition.

### Optional Components

Compositions according to the disclosure may optionally further comprise additional components useful for the particular application intended.

Direct Dyes and/or Pigments

The compositions may include one or more direct dyes and/or pigments. Including a direct dye and/or pigment may color the composition, or may provide a composition that is useful for toning the color of hair or depositing color onto the hair. As exemplary direct dyes, anionic, cationic and/or non-ionic direct dyes may be chosen.

Non-limiting exemplary direct dyes include nitrophenylenediamines, nitroaminophenols, azo dyes, anthraquinones, triarylmethane dyes, or indophenols, as well as salts thereof.

As suitable anionic (acid) direct dyes, for example, one or more compounds may be selected from the following group: Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2, D&C Brown 1, or mixtures thereof.

Non-limiting examples of suitable cationic (basic) dyes include those in the following group: Basic Blue 6 (CI No. 51,175), Basic Blue 7 (CI No. 42,595) Basic Blue 9 (CI No. 52,015), Basic Blue 26 (CI No. 44,045), Basic Blue 41 (CI No. 11,154), Basic Blue 99 (CI No. 56,059), HC Blue 15, HC Blue 16 (Blue Quat Bromide), Cationic Blue 347, Basic Brown 4 (CI No. 21,010), Basic Brown 16 (CI No. 12,250), Basic Brown 17 (CI No. 12,251), Natural Brown 7 (CI No. 75,500), Basic Green 1 (CI No. 42,040), Basic Red 2 (CI No. 50,240), Basic Red 22 (CI No. 11,055), Basic Red 51, Basic Red 76 (CI No. 12,245), Basic Violet 1 (CI No. 42,535), Basic Violet 2, Basic Violet 3 (CI No. 42,555), Basic Violet 10 (CI No. 45,170), Basic Violet 14 (CI No. 42,510), Basic Yellow 57 (CI No. 12,719), Basic Yellow 87, Basic Orange 31, or mixtures thereof.

Exemplary non-ionic direct dyes may be chosen from the following group: HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, HC Blue 15, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)-amino-2-nitrobenzene, 3-nitro-4-(2-hydroxyethyl) aminophenol, 2-(2-hydroxyethyl) amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and its salts, N,N′-bis(2-hydroxyethyl)-2-nitro-p-phenylenediamine, 2-amino-6-chloro-4-nitropheneol, 4-ethylamino-3-nitrobenzoic acid, 2-chloro-6-ethylamino-4-nitrophenol, or mixtures thereof.

As non-limiting examples of pigments, mica, carbon black, titanium oxide, chromium oxide, pigments of D&C or FD&C type and lakes thereof, such as D&C Blue No. 4, D&C Brown No. 1, FD&C Green No. 3, D&C Green No. 5, D&C Green No. 6, FD&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, FD&C Red 40 lake, D&C Violet No. 2, FD & C Blue No. 1, D&C Yellow No. 6, FD&C Yellow No. 6, D&C Yellow No. 7, Ext. D&C Yellow No. 7, D&C Yellow No. 8, D&C Yellow No. 10, or D&C Yellow No. 11, and mixtures thereof, may be chosen.

If present, the at least one direct dye and/or pigment may be present in an amount ranging from about 0.001% to about 10% by weight, such as from about 0.01% to about 10%, about 0.1% to about 10%, about 0.01% to about 5%, about 0.1% to about 5%, about 0.01% to about 5%, about 0.1% to about 5%, about 0.01% to about 4%, about 0.1% to about 4%, about 0.01% to about 3%, about 0.1% to about 3%, about 0.01% to about 2.5%, about 0.1% to about 2.5%, about 0.01% to about 2%, about 0.1% to about 2%, about 0.01% to about 1.5%, about 0.1% to about 1.5%, about 0.01% to about 1%, or about 0.1% to about 1% by weight, of the total weight of the composition.

### Active Agents

In various embodiments, compositions according to the disclosure may optionally comprise at least one active agent such as an acid or sodium hydroxide, or mixture thereof, to provide optimized strengthening benefits to the hair. Non-limiting examples of useful acids include glycolic acid, lactic acid, malic acid, tartaric acid, citric acid, ascorbic acid, mandelic acid, azelaic acid, glyceric acid, tartronic acid, gluconic acid, benzylic acid, pyruvic acid, 2-hydroxybutyric acid, salicylic acid, trichloroacetic acid, or mixtures thereof.

The acids are typically non-polymeric and may have one (mono), two (di), or three (tri) carboxylic acid groups (—COOH). The non-polymeric mono, di, and tricarboxylic acids, and/or salts thereof, typically have a molecular weight of less than about 500 g/mol, less than about 400 g/mol, or less than about 300 g/mol.

Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, and a mixture thereof.

Non-limiting examples of tricarboxylic acids include citric acid, isocitric acid, aconitric acid, propane-1,2,3-tricarboxylic acid, benzene-1,3,5-tricarboxylic acid, a salt thereof, and a mixture thereof.

If present, the total amount of the one or more acids may vary but typically ranges from about 0.0001% to about 10%, such as from about 0.0001% to about 5%, about 0.0001% to about 1%, about 0.001% to about 10%, about 0.001% to about 5%, about 0.001% to about 1% by weight, about 0.01% to about 10%, about 0.01% to about 5%, about 0.01% to about 1%, about 0.1% to about 10%, about 0.1% to about 5%, or about 0.1% to about 1% by weight, based on the total weight of the composition. For example, the total amount of the one or more acids may range from about 0.0001% to about 0.5% by weight, based on the total weight of the composition.

### Surfactants

In various embodiments, compositions according to the disclosure may optionally comprise at least one surfactant chosen from cationic surfactants, anionic surfactants, nonionic surfactants, amphoteric surfactants, and mixtures thereof.

The term “cationic surfactant” means a surfactant comprising, as ionic or ionizable groups, only cationic groups. It is understood that cationic surfactants, if present, may be included in addition to cationic conditioning agents. Non-limiting examples of cationic surfactants that may be used include polyoxyalkylenated primary, secondary, or tertiary fatty amine salts, quaternary ammonium salts, and mixtures thereof.

As quaternary ammonium salts, quaternary ammonium salts of formula (II) may be chosen:

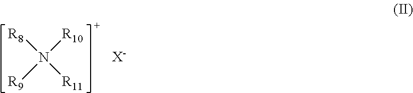

wherein:
groups R8 to R11 are independently chosen from linear or branched aliphatic groups containing from 1 to 30 carbon atoms, or an aromatic group such as aryl or alkylaryl, at least one of the groups R8 to R11 including from 8 to 30 carbon atoms, such as from 12 to 24 carbon atoms, it being possible for the linear or branched aliphatic groups to include heteroatoms such as, for example, oxygen, nitrogen, and/or sulfur, these heteroatoms not being adjacent, and halogens; and
X⁻ is an anion chosen from the group consisting of halides such as bromides, chlorides, iodides, fluorides, phosphates, acetates, lactates, (C1-C4)alkyl sulfates, (C1-C4)alkyl sulfonates or (C1-C4)alkylaryl sulfonates; C1-C30 alkyl, C1-C30 alkoxy, (C2-C6)polyoxyalkylene, C1-C30 alkylamide, (C12-C22)alkyl-(C2C6)alkylamido, (C12-C22)alkyl acetate, and C1-C30 hydroxyalkyl groups.

Mention may be made as exemplary embodiments of formula (II) of tetraalkylammonium halides, such as chlorides, for example dialkyldimethylammonium or alkyltrimethylammonium chlorides in which the alkyl group comprises from 12 to 22 carbon atoms, such as from 14 to 20 carbon atoms. By way of example, behenyltrimethylammonium chloride (behentrimonium chloride), distearyl-dimethylammonium chloride, cetyltrimethylammonium chloride (cetrimonium chloride), or benzyldimethylstearylammonium chloride may be chosen.

Mention may also be made of palmitylamidopropyltrimethylammonium or stearamidopropyldimethyl-(myristyl acetate)-ammonium halides, such as chlorides, for example the product sold under the name Ceraphyl® 70 by the company Van Dyk.

In certain embodiments, cationic surfactants of formula (II) are preferably chosen from alkyltrimethylammonium halides whose alkyl group includes from 12 to 22 carbon atoms, such as from 14 to 20 carbon atoms, may be chosen. For example, alkyltrimethylammonium chlorides, such as behenyltrimethylammonium chloride and cetyltrimethylammonium chloride, may be particularly useful.

In further embodiments, quaternary ammonium salts of imidazoline of formula (III) may be chosen:

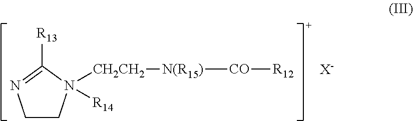

wherein:
R12 represents an alkenyl or alkyl group comprising from 8 to 30 carbon atoms, for example derived from tallow fatty acids,
R13 represents a hydrogen atom, a C1-C4 alkyl group or an alkenyl or alkyl group comprising from 8 to 30 carbon atoms,
R14 represents a C1-C4 alkyl group,
R15 represents a hydrogen atom or a C1-C4 alkyl group, and
X⁻ is an anion chosen from the group consisting of halides, phosphates, acetates, lactates, (C1-C4)alkyl sulfates, and (C1-C4)alkyl- or (C1-C4)alkylarylsulfonates.

In one exemplary embodiment of formula (III), R12 and R13 denote a mixture of alkenyl or alkyl groups comprising from 12 to 21 carbon atoms, for example derived from tallow fatty acids, R14 denotes a methyl group, and R15 denotes a hydrogen atom. Such a product is sold, for example, under the name Rewoquat® W75 or W90 by the company Evonik.

In yet further embodiments, di- or triquaternary ammonium salts of formula (IV) may be chosen:

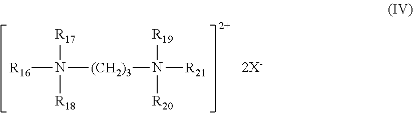

wherein:
R16 represents an alkyl group comprising from 16 to 30 carbon atoms, which is optionally hydroxylated and/or optionally interrupted with one or more oxygen atoms,
R17 represents hydrogen, an alkyl group comprising from 1 to 4 carbon atoms, or a group —(CH2)3-N⁺(R16a)(R17a)(R18a),
R16a, R17a and R18a, which may be identical or different, represent hydrogen or an alkyl group comprising from 1 to 4 carbon atoms,
R18, R19, R20, and R21, which may be identical or different, represent hydrogen or an alkyl group comprising from 1 to 4 carbon atoms, and
X⁻ is an anion chosen from the group consisting of halides, acetates, phosphates, nitrates, (C1-C4)alkyl sulfates, and (C1-C4)alkyl- and (C1-C4)alkylarylsulfonates, for example methyl sulfate or ethyl sulfate.

Such compounds are, for example, Finquat CT-P (Quaternium 89) and Finquat CT (Quaternium 75), sold by the company Finetex.

In still further embodiments, quaternary ammonium salts containing one or more ester functions, such as those of formula (V) may be chosen:

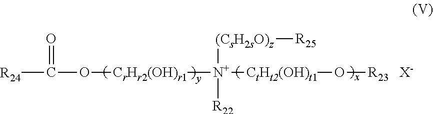

wherein:
R22 is chosen from C1-C6 alkyl groups and C1-C6 hydroxyalkyl or dihydroxyalkyl groups,
R23 is chosen from the group R26-C(═O)—; linear or branched, saturated or unsaturated C1-C22 hydrocarbon-based groups; and a hydrogen atom,
R25 is chosen from the group R28-C(═O)—; linear or branched, saturated or unsaturated C1-C6 hydrocarbon-based groups; and a hydrogen atom,
R24, R26 and R28, which may be identical or different, are chosen from saturated or unsaturated, linear or branched C7-C21 hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10, and
X⁻ is an anion,
it being understood that r2+r1=2r and t1+t2=2t, and that the sum x+y+z ranges from 1 to 15, with the proviso that when x=0 then R23 is chosen from C1-C22 hydrocarbon-based groups, and that when z=0 then R25 denotes a linear or branched, saturated or unsaturated C1-C6 hydrocarbon-based group.

In exemplary embodiments of formula (V), the alkyl groups R22 may be linear or branched, and are preferably linear. Preferably, R22 denotes a methyl, ethyl, hydroxyethyl or dihydroxypropyl group, and more particularly a methyl or ethyl group. Advantageously, the sum x+y+z ranges from 1 to 10. When R23 is a C1-C22 hydrocarbon-based groups, it may preferably comprise either from 12 to 22 carbon atoms or from 1 to 3 carbon atoms. Advantageously, R24, R26 and R28, which may be identical or different, are chosen from linear or branched, saturated or unsaturated C11-C21 hydrocarbon-based groups, and more particularly from linear or branched C11-C21 alkyl and alkenyl groups. Preferably, x and z, which may be identical or different, are equal to 0 or 1. Optionally, y is equal to 1. Preferably, r, s, and t, which may be identical or different, are equal to 2 or 3, and optionally are equal to 2.

The anion X⁻ is preferably a halide, optionally chloride, bromide, or iodide, a (C1-C4)alkyl sulfate, a (C1-C4)alkylsulfonate, or a (C1-C4)alkylarylsulfonate, a methanesulfonate, a phosphate, a nitrate, a tosylate, an anion derived from an organic acid such as an acetate or a lactate, or any other anion that is compatible with the ammonium bearing an ester function. The anion X⁻ is more particularly a chloride, a methyl sulfate, or an ethyl sulfate.

For example, the ammonium salts of formula (V) in which R22 denotes a methyl or ethyl group, x and y are equal to 1, z is equal to 0 or 1, r, s and t are equal to 2, R23 is chosen from the group R26-C(═O)—, methyl, ethyl, or C14-C22 hydrocarbon-based groups; and a hydrogen atom, R25 is chosen from the group R28-C(═O)—; and a hydrogen atom, R24, R26 and R28, which may be identical or different, are chosen from linear or branched, saturated or unsaturated C13-C17 hydrocarbon-based groups, and preferably from linear or branched, saturated or unsaturated C13-C17 alkyl and alkenyl groups, may be chosen. Advantageously, the hydrocarbon-based groups are linear.

Among the compounds having formula (V), mention may be made of salts, especially the chloride or methyl sulfate salts, of diacyloxyethyldimethylammonium, diacyloxyethylhydroxyethylmethylammonium, monoacyloxyethyldihydroxyethylmethyl-ammonium, triacyloxyethylmethylammonium or monoacyloxyethylhydroxyethyldimethyl-ammonium, and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures especially of plant or animal origin, or by transesterification of the methyl esters thereof. This esterification may be followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The one or more cationic surfactants may be chosen from, for example, a mixture of quaternary ammonium monoester, diester and triester salts with a weight majority of diester salts. Use may also be made of the ammonium salts containing at least one ester functional group that are described in patents U.S. Pat. Nos. 4,874,554 and 4,137,180. Use may also be made of behenoylhydroxypropyl-trimethylammonium chloride, for example, sold by the company Kao under the name Quartamin BTC 131. Preferably, the ammonium salts containing at least one ester function contain two ester functions.

In certain embodiments according to the disclosure, for example skin cleansing compositions, the compositions may comprise one or more anionic surfactants. The term “anionic surfactant” means a surfactant comprising, as ionic or ionizable groups, only anionic groups. These anionic groups may optionally be chosen from the groups CO₂H, CO₂—, SO₃H, SO₃—, OSO₃H, OSO₃⁻ O₂PO₂H, O₂PO₂H and O₂PO₂²⁻.

Non-limiting examples of anionic surfactants include alkyl sulfates, alkyl ether sulfates, acyl isethionates, acyl glycinates, acyl taurates, acyl amino acids, acyl sarcosinates, sulfosuccinates, sulfonates, and a mixture thereof, wherein the alkyl and acyl groups of all these compounds comprise from 6 to 24 carbon atoms. In some cases, anionic sulfate surfactants may be excluded from the one or more anionic surfactants. In such cases, the one or more anionic surfactants may be selected from the group consisting of acyl isethionates, acyl glycinates, acyl taurates, acyl amino acids, acyl sarcosinates, sulfosuccinates, sulfonates, and mixtures thereof, wherein the alkyl and acyl groups of all these compounds comprise from 6 to 24 carbon atoms.

The anionic surfactant(s) that may be used may be alkyl sulfates, alkyl ether sulfates, alkylamido ether sulfates, alkylaryl polyether sulfates, monoglyceride sulfates, alkylsulfonates, alkylamide sulfonates, alkylarylsulfonates, alpha-olefin sulfonates, paraffin sulfonates, alkylsulfosuccinates, alkyl ether sulfosuccinates, alkylamide sulfosuccinates, alkyl sulfoacetates, acylsarcosinates, acylglutamates, alkylsulfosuccinamates, acylisethionates and N-acyltaurates, salts of alkyl monoesters and polyglycoside-polycarboxylic acids, acyllactylates, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units. For example, the anionic surfactant may be chosen from sodium olefin sulfonates, e.g. sodium C14-C16 olefin sulfonate.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates, and C₆-C₂₄ alkyl polyglycoside-sulfo succinates.

When the anionic surfactant(s) are in salt form, they may be chosen especially from alkali metal salts such as the sodium or potassium salt and preferably the sodium salt, ammonium salts, amine salts and in particular amino alcohol salts, or alkaline-earth metal salts such as magnesium salts.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts. Alkali metal or alkaline-earth metal salts, such as sodium or magnesium salts, may be used.

Use is also made of (C₆-C₂₄)alkyl sulfates, (C₆-C₂₄)alkyl ether sulfates, which are optionally ethoxylated, comprising from 2 to 50 ethylene oxide units, and a mixture thereof, in particular in the form of alkali metal salts or alkaline-earth metal salts, ammonium salts or amino alcohol salts. In certain embodiments, the anionic surfactant(s) are chosen from (C₁₀-C₂₀)alkyl ether sulfates, for example sodium lauryl ether sulfate.

Useful non-ionic surfactant(s) can be, for example, selected from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀—C₁₄)acyl-aminopropylmorpholine oxides; and a mixture thereof.

The nonionic surfactants may optionally be chosen from polyoxyalkylenated or polyglycerolated nonionic surfactants. The oxyalkylene units are optionally oxyethylene or oxypropylene units, or a mixture thereof. Examples of oxyalkylenated nonionic surfactants that may be mentioned include: oxyalkylenated (C₈-C₂₄)alkyl-phenols, saturated or unsaturated, linear or branched, oxyalkylenated C₈-C₁₀ alcohols, saturated or unsaturated, linear or branched, oxyalkylenated C₈-C₁₀ amides, esters of saturated or unsaturated, linear or branched, C₈-C₁₀ acids and of polyethylene glycols, polyoxyalkylenated esters of saturated or unsaturated, linear or branched, C₈-C₁₀ acids and of sorbitol, saturated or unsaturated, oxyalkylenated plant oils, condensates of ethylene oxide and/or of propylene oxide, inter alia, alone or as mixtures.

As examples of polyglycerolated nonionic surfactants, polyglycerolated C₈-C₄₀ alcohols are preferably used. In particular, the polyglycerolated C₅-C₄₀ alcohols correspond to the following formula (VI):

RO—[CH₂—CH(CH₂OH)—O]ₘ—H or RO—[CH(CH₂OH)—CH₂O]ₘ—H (VI)

in which R represents a linear or branched C₅-C₄₀ and preferably C₈-C₁₀ alkyl or alkenyl radical, and m represents a number ranging from 1 to 30, and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

According to one embodiment, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and a mixture thereof.

Preferably, the nonionic surfactant may be a nonionic surfactant with an HLB of 18.0 or less, such as from 4.0 to 18.0, more preferably from 6.0 to 15.0 and furthermore preferably from 9.0 to 13.0. The HLB is the ratio between the hydrophilic part and the lipophilic part in the molecule.

In some case, the nonionic surfactant is a fatty alkanolamide. Non-limiting examples of fatty alkanolamides that may be used include cocamide MEA, cocamide DEA, soyamide DEA, lauramide DEA, oleamide MIPA, stearamide MEA, myristamide DEA, stearamide DEA, oleylamide DEA, tallowamide DEA lauramide MIPA, tallowamide MEA, isostearamide DEA, isostearamide MEA, or mixtures thereof.

Non-limiting examples of amphoteric surfactants useful in the compositions include, for example, optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C8-C20)alkylbetaines, sulfobetaines, (C8-C20)alkylsulfobetaines, (C8-C20)alkylamido(C1-C6)alkylbetaines, such as cocamido-propylbetaine, and (C8-C20)alkylamido(C1-C6)alkylsulfobetaines, and mixtures thereof. For example, mention may be made of compounds classified under the INCI names sodium cocoamphoacetate, sodium lauroamphoacetate, sodium caproamphoacetate and sodium capryloamphoacetate.

Other compounds that may be chosen include disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

Examples that may be mentioned include the cocoamphodiacetate sold by the company Rhodia under the trade name Miranol® C2M Concentrate, the sodium cocoamphoacetate sold under the trade name Miranol® Ultra C 32 and the product sold by the company Chimex under the trade name CHIMEXANE HA. In certain exemplary embodiments, the amphoteric surfactants are chosen from (C8-C20)alkylbetaines such as the one known under the INCI names coco-betaine, (C8-C20)alkylamido(C1-C6)alkylbetaines such as the one known under the INCI name cocamidopropylbetaine, and mixtures thereof.

If present, the total amount of the one or more surfactants can range from about 0.001% to about 10% by weight, relative to the total weight of the composition, including all ranges and subranges therebetween. For example, the total amount of the one or more surfactants can range from about 0.001% to about 5%, such as from about 0.001% to about 4%, about 0.001% to about 3%, about 0.001% to about 2%, about 0.001% to about 1%, about 0.001% to about 0.5%, about 0.01% to about 5%, about 0.01% to about 4%, about 0.01% to about 3%, about 0.01% to about 2%, about 0.01% to about 1%, about 0.01% to about 0.5%, about 0.1% to about 5%, about 0.1% to about 4%, about 0.1% to about 3%, about 0.1% to about 2%, about 0.1% to about 1%, or about 0.1% to about 0.5%, by weight, relative to the total weight of the hair treatment composition.

Compositions according to the disclosure may optionally comprise any auxiliary component suitable for use in cosmetic compositions. Such components may include, but are not limited to, dyes/pigments in addition to those listed above, ceramides, thickening agents, film forming agents or polymers, humectants and moisturizing agents, fatty substances other than fatty alcohols, emulsifying agents, fillers, structuring agents, propellants, shine agents, antioxidants or reducing agents, penetrants, sequestrants, fragrances, buffers, dispersants, plant extracts, volatile or non-volatile, modified or unmodified silicones, preserving agents, opacifiers, sunscreen agents, vitamins, pH adjusting agents, and antistatic agents. In certain embodiments, the compositions are free or substantially free of silicones.

Optional auxiliary components may be present in an amount ranging from about 0.0001% to about 15% by weight, relative to the total weight of the composition.

### II. Systems and Kits

Embodiments of the present disclosure further provide systems and kits comprising any of the compositions described herein. In various embodiments, the systems and kits include an aerosol device containing a low-viscosity composition comprising vegetable oil, at least one fatty alcohol, at least one cationic conditioning agent, and at least one propellant, and a dispensing means for dispensing the content of the container, such as a diffuser or spray device which the user can press to make the product come out. In the aerosol device, the composition is generally in the form of a low-viscosity fluid emulsion, also referred to as a “juice” or “juice phase,” suitable for forming a foam when aerosolized. As such, aerosol devices comprising compositions according to the disclosure are also disclosed.

Various aerosol devices may be employed for use according to the disclosure. By way of non-limiting example, typical aerosol hair spray devices expel the juice phase in the form of a mist of dispersed droplets. The juice phase is primarily comprised of the hair styling compositions of the instant disclosure, excluding the propellant(s). Small amounts of propellant(s) or other materials from the vapor phase may be added to the juice phase, for example, these components may be partially dispersed or solubilized in the juice phase. Likewise, the vapor phase is primarily comprised of propellant(s). Small amounts of juice phase (or components of the juice phase) may be present in the vapor phase to the extent that the juice phase (or components of the juice phase) are dispersed or solubilized in the vapor phase. In systems and kits according to the disclosure, the aerosol device may be any type of container or canister that is configured to dispense its liquid contents as a foam. Such container or canister may be in any size, color, or shape. For example, a system or kit as disclosed herein may be any container or a canister containing a composition according to the disclosure, and equipped with a nozzle designed to aerosolize the composition to form a foam. The container or canister may have a button to trigger or control dispensing the aerosol contained therein. The aerosol containers may also be made of any materials suitable for cosmetic use.

The total amount of propellant used in an aerosol device according to the disclosure may vary. In some cases, an aerosol device may include propellant in an amount ranging from about 1% to about 95%, based on the total weight of the hair compositions and propellant (i.e., the total weight of the content inside the aerosol device). In some cases, the total amount of propellant may be about 1% to about 90%, about 1% to about 50%, about 1% to about 20%, about 5% to about 90%, about 5% to about 50%, or about 5% to about 20% by weight. For example, the aerosol device may include propellant in an amount of about 5%, such as about 6%, and juice phase in an amount of about 95%, such as about 94%, based on the total weight of the juice phase and propellant.

Kits according to the disclosure may comprise an aerosol device as disclosed herein. For example, a kit may comprise an aerosol device containing a low-viscosity composition comprising vegetable oil, at least one fatty alcohol, at least one cationic conditioning agent, and at least one propellant. In various embodiments, a kit may comprise an aerosol device comprising a composition according to the disclosure, and one or more skin or hair treatment compositions housed in a separate container. For example, the one or more hair treatment compositions housed in a separate container may include but are not limited to a hair-treatment rinse, a shampoo, a hair-color-toning composition, a hair lightening composition, a hair coloring composition, a hair relaxing composition, a hair straightening composition, or a hair waving composition.

### III. Methods

Methods according to the disclosure comprise treating, caring for, or conditioning keratin substrates with the compositions described herein. The methods generally comprise applying any of the disclosed compositions to the keratin substrates, e.g. to the hair or skin, and optionally subsequently rinsing the compositions.

By way of non-limiting example, the methods may include use of the compositions as a hair conditioner, a pre- or post-treatment for hair, a rinse-off or leave-on mask treatment for hair, or the like. For example, the composition may be a wash-out or leave-on hair conditioner, and the methods may be directed to conditioning the hair by applying the aerosolized compositions to the hair. As further examples, the methods may include use of the compositions as a toning rinse-off or leave-on mousse for hair color.

Without limitation, methods of treating hair according to the disclosure may include applying an aerosol foam composition according to the disclosure to hair (wet, damp, or dry hair), allowing the composition to remain on the hair for a desired amount of time, and optionally rinsing the composition from the hair. The composition may be applied to the hair before, during, or after other hair compositions (e.g., a shampoo, a conditioner, a mask, a cream, a lotion, a gel, any other chemical treatment, etc.). If desired, the hair may be heated while the composition is on the hair.

In yet further embodiments, the methods may include using the compositions disclosed herein for skin care or treatment. In some embodiments, the methods include using compositions disclosed herein for cleansing, caring for, or moisturizing skin. The methods may include applying an aerosol foam composition according to the disclosure to skin, allowing the composition to remain on the skin for a desired amount of time, and optionally rinsing the composition from the skin.

When using the compositions as a rinse-off composition, the composition may be allowed to remain on the keratin substrate (e.g. skin or hair) for any desired amount of time, for example from about a few seconds (e.g. about 10, 20, or 30 seconds) to about 1, 2, 3, 5, 10, 20, or 30 minutes, or longer.

Having described the many embodiments of the present invention in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise.

The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

Throughout the disclosure, if the term “a mixture thereof,” including variants, is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

As used herein, the terms “applying a composition onto keratin fibers” and “applying a composition onto hair” and variations of these phrases are intended to mean contacting the keratin fibers or hair, with at least one of the compositions of the invention, in any manner. It may also mean contacting the keratin fibers or hair in an effective amount

Unless otherwise indicated, all percentages herein are by weight, relative to the weight of the total composition.

As used herein, “cosmetic composition” encompasses many types of compositions for application to keratinous materials such as skin or hair, for example, hair lotions, hair creams, hair gel creams, hair conditioners, hair masques (masks), etc., which can be used either as leave-on or rinse-off treatments or products.

As used herein, the term “organic” means a material that is produced substantially without or essentially without the use of synthetic materials. The term “substantially without” or “essentially without” as used herein means the specific material may be used in a manufacturing process in small amounts that do not materially affect the basic and novel characteristics of the compositions according to the disclosure. The term “substantially without” or “essentially without” as used herein may also mean that the specific material is not used in a manufacturing process but may still be present in a raw material that is included in the composition.

As used herein, the “salts” referred to throughout the disclosure may include salts having a counter-ion such as an alkali metal, alkaline earth metal, or ammonium counterion. This list of counterions, however, is non-limiting.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “surfactants” includes salts of the surfactants even if not explicitly stated.

As used herein, the term “synthetic” means a material that is not of natural origin. The term “natural” means a material of natural origin, such as derived from plants, which also cannot be subsequently chemically or physically modified.

### EXAMPLES

The following examples are intended to be non-limiting and explanatory in nature only. In the Examples, amounts are expressed in percentage by weight (wt %) of active materials.

### Example 1—Hair Conditioner Compositions

Three inventive hair compositions and two comparative hair compositions were prepared according to the formulations set forth in Table 1 below.

TABLE 1

| | Composition | Composition | Composition | Composition | Composition |
|---|---|---|---|---|---|
| | 1A | 1B | 1C | 1D | 1E |
| | (Inventive | (Inventive | (Inventive | (Comparative | (Comparative |
| INCI NAME | Composition) | Composition) | Composition) | Composition) | Composition) |
| SURFACTANT | 0.14 | 0.14 | 0.14 | 0.15 | 0.15 |
| (glyceryl stearate) | | | | | |
| CATIONIC | 2.35 | 2.35 | 2.35 | 2.50 | 2.50 |
| CONDITIONING | | | | | |
| AGENT | | | | | |
| (brassicamidopropyl | | | | | |
| dimethylamine) | | | | | |
| LIQUID FATTY | 0.47 | 0.47 | 0.47 | 0.50 | 0.50 |
| ALCOHOL | | | | | |
| (octyldodecanol) | | | | | |
| SOLID FATTY | 2.82 | 2.82 | 2.82 | 3.00 | 3.00 |
| ALCOHOL | | | | | |
| (cetearyl alcohol) | | | | | |
| PROPELLANTS | 6.00 | 6.00 | 6.00 | — | — |
| (propane + isobutane) | | | | | |
| COCOS NUCIFERA | 0.94 | 0.94 | 0.94 | 1.00 | 1.00 |
| (COCONUT) OIL | | | | | |
| ADDITIONAL | 0.99 | 1.46 | 1.46 | 1.06 | 0.90 |
| COMPONENTS | | | | | |
| (plant extracts, | | | | | |
| preservatives, vitamins, | | | | | |
| thickeners, fragrance, | | | | | |
| pigments) | | | | | |
| tartaric acid | 0.33 | 0.33 | 0.33 | 0.35 | 0.35 |
| SOLVENT (water + | QS | QS | QS | QS | QS |
| non-aqueous solvents) | | | | | |

Each of Compositions 1A-1E was prepared by adding the solvent and stirring, then adding the acid to obtain a first mixture, which was then heated. Once the first mixture reached a temperature of about 70-7500C, the fatty alcohols, cationic conditioning agents, coconut oil, glycols, and additional components were added to the first mixture to obtain a second mixture, which was homogenized at about 1500-2000 RPM for about 10 minutes and then cooled to room temperature. Once the second mixture was cooled to room temperature, optional ingredients were added to obtain a finished bulk of mixture. Finally, for Compositions 1A-1C, the propellants were added and the composition was aerosolized.

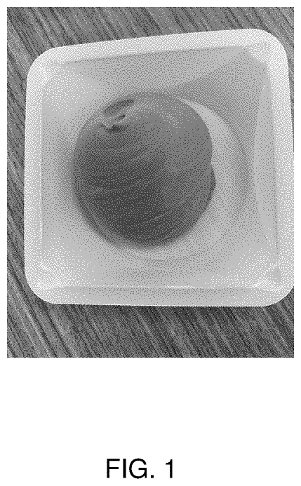

Inventive Compositions 1A-1C were low-viscosity compositions which were easily aerosolized into dense, smooth, luxurious, stable foams. This can be seen in the photographic image in FIG. 1, which is the hair-color-toning foam prepared in Example 1C.

### Example 2—Comparison of Properties of Compositions and Treated Hair

A comparative study was conducted to demonstrate the properties and performance of aerosol foam compositions according to the disclosure. After Composition 1A was aerosolized to form a foam hair conditioner, it was tested and compared with the non-aerosolized hair conditioner Composition 1 D in a salon half-head study performed on the hair of 10 volunteers. The hair of the volunteers was medium to long in length, having average diameter, curl pattern of 1-3, and sensitized level of 3-4.

The hair of the volunteers was first cleansed with a commercially available shampoo. While the hair was still damp, an amount of Composition 1A needed to condition the hair was applied to one-half of the head of hair and distributed evenly with fingers. The same amount of Composition 1 D was then applied to the other half of the head of hair and distributed evenly with fingers. Both Compositions 1A and 1D were allowed to remain on the hair for about 5 minutes. After rinsing the compositions from the hair, the hair was combed, dried, and styled.

During application, Composition 1A was rated better for consistency, melting sensation in hands, coating effect, and absorption than Composition 1 D.

During and after combing, drying, and styling the hair, the hair treated with Composition 1A was rated better for surface effect, dry end results, smooth hair feel, ease of shaping, and dryness than Composition 1 D.

In general, when compared to the comparative non-aerosolized conditioner composition, the aerosolized foam conditioner composition according to the disclosure had better application properties and left hair with improved sensorial properties than the comparative non-aerosolized composition.

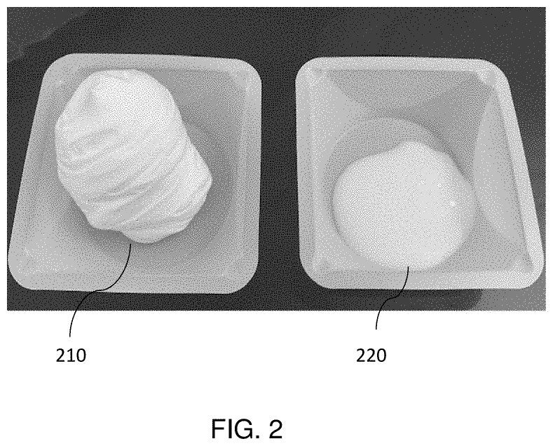

FIG. 2 is a photographic image showing the foam 210 formed by Composition 1A after being aerosolized in comparison with the cream conditioner 220 of Composition 1D in a similar amount. As seen in FIG. 2, the foam 210 formed by Composition 1A is compact and dense and does not collapse. The foam 210 also does not look “airy” or “whipped,” and is surprisingly stable.

### Example 3—Properties of Aerosol Compositions Comprising Vegetable Oil

As described, high levels of vegetable oil are not typically included in traditional emulsion cosmetic compositions. Therefore, a comparative study was conducted to demonstrate the impact of including vegetable oil in the disclosed aerosol foam compositions.

The study was done by comparing the appearance and rheological characteristics of two compositions prepared based on the same formulation, with the only difference being whether or not vegetable (coconut) oil was included. Composition 1A in Table 1 was compared with a comparative composition including all the same ingredients except coconut oil (Composition 1A′). In Composition 1A′, the coconut oil was replaced with the same amount of water.

The viscosities of Compositions 1A and 1A′, as emulsions, were measured using a Rheomat 180 viscometer at about 25° C./spindle 2 (Composition 1A, 320) or 3 (Composition 1A′, 310) for 30 seconds. The viscosity of Composition 1A was found to be within a range of about 10 to about 30 DU (“Deflection Units”) when measured at about 25° C., 200 RPM, 30 seconds on a spindle 2 of the viscometer. The viscosity of Composition 1A′ was found to be at least about 10 DU when measured at 25° C., 200 RPM, 30 seconds on a spindle 3 of the viscometer, which indicates an increase in thickness.

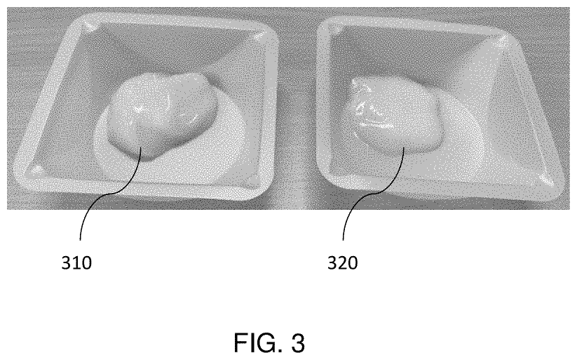

A comparison of Compositions 1A and 1A′ prior to being aerosolized is shown in FIG. 3. As can be seen, Composition 1A, 320 is noticeably less viscous than Composition 1A′, 310. This difference suggests that Composition 1A′, which is thicker, will be more difficult to aerosolize, while Composition 1A is more fluid, indicating that it will be aerosolized more easily. Without coconut oil, Composition 1A′ resulted in a product that was too thick and could not be aerosolized into a viable product, and could not be dispensed properly.

This comparison thus demonstrated the benefit of using vegetable oil in aerosol foam compositions according to the disclosure.

### Example 4—Comparison to Compositions with Silicones

A comparative study was conducted to demonstrate that silicone-based compositions, such as high silicone hair masks, do not aerosolize well. Two exemplary compositions were prepared by mixing the ingredients according to the formulations set forth in Table 2 below.

TABLE 2

| | Composition | Composition |
|---|---|---|
| | 4A | 4B |
| | (Comparative | (Comparative |
| INCI NAME | Composition) | Composition) |
| BEHENTRIMONIUM | 3.75 | 4 |
| CHLORIDE | | |
| OTHER INGREDIENTS | 1.63 | 2.52 |
| WATER | 77.12 | 85.98 |
| LIQUID FATTY ALCOHOL | 7 | 3.5 |
| ESTER | 2 | — |
| SILICONE | 8.5 | 4 |

The viscosities of Compositions 4A and 4B were measured using a Rheomat 180 viscometer at about 25° C. for 30 seconds at 200 RPM on a spindle 3 of the viscometer. Both Compositions 4A and 4B have a viscosity range of about 30-85 DU on the Spindle 3. Such viscosity and combination of raw materials impeded the compositions' ability of actuating effortlessly into a stable, compact foam. The compositions could not be aerosolized well.

### Example 5—Evaluation of Stability of Aerosolized Foams

An evaluation was performed to study the stability of a foam conditioner composition according to the disclosure (Composition 1A in Table 1) (“Inventive Foam”), relative to a commercially available foam conditioner (“Comparative Foam”) having the following components:

TABLE 3
Commercially Available Foam Conditioner

WATER
PROPELLANTS
CETEARYL ALCOHOL
DIMETHYL ETHER
STEARAMIDOPROPYL DIMETHYLAMINE
BEHENTRIMONIUM CHLORIDE
FRAGRANCE
DIMETHICONE
DMDM HYDANTOIN
DIPROPYLENE GLYCOL
LACTIC ACID
PVP
DISODIUM EDTA
AMODIMETHICONE
BUTYLENE GLYCOL
IODOPROYNYL BUTYLCARBAMATE
PEG-7 PROPYLHEPTYL ETHER
CETRIMONIUM CHLORIDE
COLLAGEN AMINO ACIDS

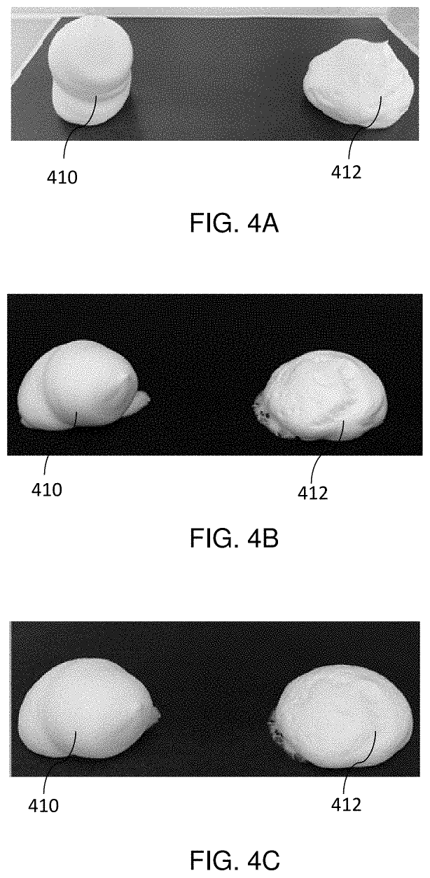

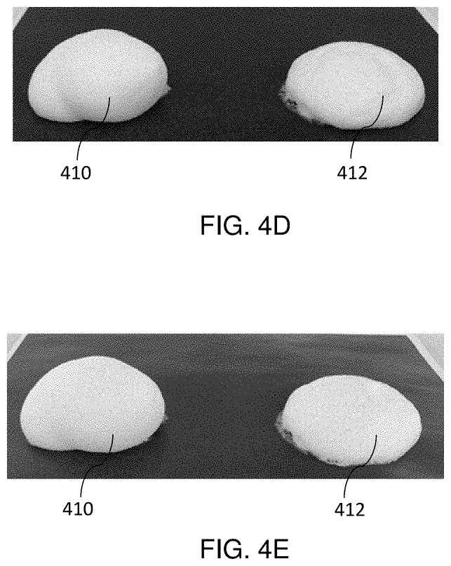

The study was conducted by dispensing approximately equal amounts of conditioner compositions to form foams, and observing the behavior of the foams for a period of time. As can be seen in FIG. 4A, at time (t)=0, i.e. immediately after aerosolization, the Inventive Foam 410 is compact, dense, and holds its shape, while the Comparative Foam 412 is airy and already starting to collapse. In FIG. 4B, at t=10 minutes, i.e. 10 minutes after aerosolization, the Inventive Foam 410 is still dense, whereas the Comparative Foam 412 is starting to lose its properties as a foam. In FIG. 4C, at t=30 minutes, Comparative Foam 410 is essentially unchanged, while Comparative Foam 412 continues to lose its foam properties. The photographs in FIG. 4D (t=45 minutes) and 4E (t=60 minutes) demonstrate that the Inventive Foam 410 maintains is shape and appears dense even after one hour, whereas the Comparative Foam 412 continued to dissolve and lose its characteristics as a foam.

This Example demonstrates that the foam compositions according to the disclosure are stable compared to similar conventional foam products.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A composition for treating keratinous substrates comprising:
(a) at least one vegetable oil;
(b) at least one liquid fatty alcohol;
(c) at least one cationic conditioning agent; and
(d) at least one propellant;
wherein the composition is in the form of a fluid emulsion and is free or substantially free of a silicone.

2. The composition of claim 1, wherein the at least one vegetable oil is present in an amount ranging from about 0.5% to about 15% by weight, relative to the total weight of the composition.

3. The composition of claim 1, wherein the at least one vegetable oil comprises coconut oil.

4. The composition of claim 1, wherein the at least one liquid fatty alcohol is chosen from C8-C30 liquid fatty alcohols.

5. The composition of claim 1, wherein the at least one propellant is present in an amount ranging from about 0.01% to about 10% by weight, relative to the total weight of the composition.

6. The composition of claim 1, wherein the at least one cationic conditioning agent is chosen from amidoamines of formula (I) or salts thereof:
RCONHR′N(R″)₂ (I)
wherein:
R is a linear or branched, acyclic or cyclic, saturated or unsaturated, aliphatic or aromatic, substituted or unsubstituted hydrocarbon radical containing at least 6 carbon atoms, optionally;
R′ is a linear or branched, acyclic or cyclic, saturated or unsaturated, aliphatic or aromatic, substituted or unsubstituted divalent hydrocarbon radical containing less than 6 carbon atoms; and
R″ is H or a hydrocarbon radical containing less than 6 carbon atoms.

7. The composition of claim 1, wherein the at least one cationic conditioning agent is present in an amount ranging from about 0.01% to about 15% by weight, based on the total weight of the composition.

8. The composition of claim 1, further comprising:
(e) at least one solid fatty alcohol.

9. The composition of claim 8, wherein the total amount of the at least one solid fatty alcohol is present in an amount ranging from about 0.01% to about 15% by weight, based on the total weight of the composition.

10. The composition of claim 1, further comprising at least one component chosen from active agents, direct dyes, pigments, surfactants, or mixtures thereof.

11. A system comprising an aerosol device containing:
(a) at least one vegetable oil;
(b) at least one liquid fatty alcohol;
(c) at least one cationic conditioning agent;
(d) at least one propellant;
(e) optionally at least one solid fatty alcohol; and
(f) optionally at least one additional component chosen from active agents, direct dyes, pigments, surfactants, or mixtures thereof;
wherein the composition is in the form of a fluid emulsion and is free or substantially free of a silicone, and
wherein the aerosol device comprises a means for dispensing the content of the container.

12. The system of claim 11, wherein the at least one vegetable oil is present in an amount ranging from about 0.5% to about 15% by weight, relative to the total weight of the composition.

13. The system of claim 11, wherein the at least one vegetable oil comprises coconut oil.

14. The system of claim 11, wherein the at least one liquid fatty alcohol is chosen from C8-C30 liquid fatty alcohols.

15. The system of claim 11, wherein the at least one propellant is present in an amount ranging from about 0.01% to about 10% by weight, relative to the total weight of the composition.

16. The system of claim 11, wherein the at least one cationic conditioning agent is chosen from amidoamines of formula (I) or salts thereof:
RCONHR′N(R″)₂ (I)
wherein:
R is a linear or branched, acyclic or cyclic, saturated or unsaturated, aliphatic or aromatic, substituted or unsubstituted hydrocarbon radical containing at least 6 carbon atoms, optionally;
R′ is a linear or branched, acyclic or cyclic, saturated or unsaturated, aliphatic or aromatic, substituted or unsubstituted divalent hydrocarbon radical containing less than 6 carbon atoms; and
R″ is H or a hydrocarbon radical containing less than 6 carbon atoms.

17. The system of claim 11, wherein the at least one cationic conditioning agent is present in an amount ranging from about 0.01% to about 15% by weight, based on the total weight of the composition.

18. The system of claim 11, wherein the at least one fatty alcohol is present in an amount ranging from about 0.01% to about 15% by weight, based on the total weight of the composition.

19. A method of treating, caring for, or conditioning a keratin substrate comprising:
applying to the keratin substrate an aerosolized foam composition, wherein the aerosolized foam composition comprises:
(a) at least one vegetable oil;
(b) at least one liquid fatty alcohol;
(c) at least one cationic conditioning agent;
(d) at least one propellant;
(e) optionally at least one solid fatty alcohol; and
(f) optionally, at least one additional component chosen from active agents, direct dyes, pigments, surfactants, or mixtures thereof,
wherein the aerosolized foam composition is free or substantially free of a silicone; and
optionally rinsing the composition from the keratin substrate.

20. A kit comprising:
an aerosol container containing a first composition, the first composition comprising:
(a) at least one vegetable oil;
(b) at least one liquid fatty alcohol;
(c) at least one cationic conditioning agent;
(d) at least one propellant;
(e) optionally at least one solid fatty alcohol; and
(f) optionally, at least one at least one additional component chosen from active agents, direct dyes, pigments, surfactants, or mixtures thereof, and
wherein the first composition is in the form of a fluid emulsion and is free or substantially free of a silicone; and
a second container comprising a second composition for treating, caring for, or conditioning keratin substrates.
